(19) United States
(12) Patent Application Publication
(10) Pub. No.: US 2026/0034013 A1
(43) Pub. Date: Feb. 5, 2026

(54) EXOSKELETON

(71) Applicant: Elysium Industries UG, Gilching (DE)

(72) Inventors: Nikolaus Batge, Gilching (DE); Jakub Devecka, Gilching (DE)

(21) Appl. No.: 18/867,747

(22) PCT Filed: May 17, 2023
(86) PCT No.: PCT/EP2023/063395

§ 371 Date: Nov. 20, 2024

(30) Foreign Application Priority Data
May 20, 2022 (DE) 10 2022 112 709.5

(51) Int. Cl. A61H 1/02; A61F 5/01; A61H 3/00

(52) U.S. Cl. CPC A61H 1/024; A61F 5/0125; A61H 3/00; A61F 2005/0155; A61F 2005/0167; A61F 2005/0179; A61H 2201/0192; A61H 2201/1215; A61H 2201/1246; A61H 2201/14; A61H 2201/164; A61H 2201/1673; A61H 2201/1676

(57) ABSTRACT

The invention relates to an exoskeleton that includes upper and lower structural parts pivotably interconnected by means a joint; a deflection guide arranged in front of the joint; an elongate spring element extending between upper and lower connection points and which is guided over the deflection guide. A deflection-guide positioning device in the form of a device for resiliently positioning the deflection guide is provided and is designed such that the deflection guide can be resiliently positioned in the direction in which the spacing increases and reduces between the deflection guide and the joint, in order to dynamically adapt the degree of relief and/or support; or wherein the deflection-guide positioning device is in the form of a device for moving the deflection guide and is designed such that the spacing between the deflection guide and the joint, can be adjusted in order to adjust the degree of relief and/or support.

This application is a national stage application of PCT Patent Appln. No. PCT/EP2023/063395 filed Nov. 23, 2023, which claims priority to DE Patent Appln. No. 10 2022 112 709.5 filed May 20, 2022, which are herein incorporated by reference.

### BACKGROUND OF THE INVENTION

### 1. Technical Field

The present invention relates to an exoskeleton for relieving and/or supporting a human knee joint.

### 2. Background Information

Gait-supporting exoskeletons, which are attached to a human thigh and a human lower leg and are intended to support the movement of the human knee joint and relieve the strain on the human knee joint when walking, often have considerable actuator technology, sensor technology and control/regulation systems to ensure support and relief in each situation. This makes such exoskeletons expensive to manufacture and complex to handle and use. In addition, such exoskeletons require considerable energy to control the actuators, sensors and control/regulation systems and to ensure the required joint torques.

For example, a knee orthosis with a thigh structure, a lower leg structure and an actuator unit between the thigh and lower leg structure is known from EP 2 276 433 A2. An effective ankle moment is determined within the orthosis, and the behavior of the actuator unit is changed as a function of the ankle moment and/or the knee angle.

### SUMMARY

It is therefore an object of the present invention to provide an exoskeleton with which effective relief and/or support of a human knee joint can be achieved, but which can be manufactured and operated more cost-effectively and which is easy to handle.

This problem is solved by the subject matter of independent claim 1. Advantageous further developments result from the dependent claims.

An exoskeleton according to the invention is understood to be an external support structure for a human knee joint, which is attached with its upper structural part to a human thigh and with its lower structural part to a human lower leg and which relieves and/or supports the human knee joint during operation. Such an exoskeleton can also be used in the medical field, where it is referred to as an orthosis.

According to the invention, the exoskeleton is provided with a deflection guide arranged in front of the joint and an elongate spring element, in particular an elongate elastomer spring, an elastic band, or a band-spring combination, which extends between an upper connection point, which is arranged in particular in a lateral, front region of the upper structural part, and a lower connection point, which is arranged in particular in a lateral, front region of the lower structural part, and which is guided over the deflection guide, which defines a deflection point or a deflection path for the elongate spring element.

According to an insight underlying the invention, by changing the distance between the deflection guide and the joint, as viewed essentially in the direction from the joint to the front, the spring action and in particular the characteristic curve of the spring action can be adjusted so that the appropriate moment is applied to the exoskeleton in each case, i.e. in order to achieve relief and support by the elongate spring element that is individually adapted to the respective human knee joint. A deflection-guide positioning device is provided for this purpose.

According to a first variant of the invention, the deflection-guide positioning device is designed as a device for resilient positioning of the deflection guide and is arranged such that the deflection guide can be positioned resiliently in the direction of increasing the distance and decreasing the distance between the deflection guide and the joint, as seen essentially in the direction from the joint to the front, for dynamic adjustment of the degree of relief and/or support.

According to a second variant of the invention, the deflection-guide positioning device is configured as a device for adjusting the deflection guide and is configured such that the distance between the deflection guide and the joint, as viewed substantially in the forward direction from the joint, is adjustable for adjusting the degree of relief and/or support.

In the second variant of the invention, the user can be provided with instructions for which desired operating mode the deflection-guide positioning device is to be locked in which position, so that the user can adjust the deflection-guide positioning device to the corresponding position and the corresponding distance to the joint by means of the corresponding spring or the adjustment cable.

Due to the basic kinematic design of an exoskeleton according to the invention, very high forces can be achieved in the elongate spring element through comparatively small retaining forces of the deflection-guide positioning device, thereby achieving the desired torque at the joint of the exoskeleton.

In the first variant of the invention, the distance between the deflection guide and the joint changes when the upper and lower structural parts and thus the human upper and lower leg are pivoted in relation to each other. This results in a dynamic adjustment of the acting moment, and thus the degree of relief and/or support is dynamically adjusted.

In the second variant of the invention, the distance between the deflection guide and the joint, as seen essentially in the direction from the joint to the front, is set to a desired distance by the deflection-guide positioning device, which is designed as a device for adjusting the deflection guide. When the exoskeleton is used, the relief and/or support is then provided based on the moments that arise when the upper and lower structural parts pivot in relation to each other and the corresponding pivoting of the human upper and lower leg in relation to each other from the respective moment that is applied with respect to the joint of the exoskeleton and is generated by the spring action of the elongate spring element.

It is recommended to adapt the exoskeleton according to the invention to the respective human knee joint before use by selecting or adjusting the elongate spring element, in particular by suitable selection of the elongate spring element and/or by adjusting the spring characteristic curve and/or the spring action and/or the spring pretension of the elongate spring element, and/or by adjusting the upper and lower connection point of the elongate spring element.

The moment adjustment by the deflection-guide positioning device can be achieved by the corresponding design of the same, according to the embodiments explained in detail below.

With the exception of the embodiment with actuator described below, the exoskeleton according to the invention does not require any actuators, sensors or electronic control/regulation systems and can therefore be manufactured and operated at low cost, especially since no electrical energy is required for operation.

In the embodiment with actuator described below, the manufacturing costs and the costs in operation are also comparatively low because the actuator does not have to apply the actual gait-supporting forces but, in the first variant of the invention, only has to resiliently position the deflection guide or, in the second variant of the invention, only has to adjust the deflection guide once to the desired position. For this purpose, the actuator can be designed to be correspondingly small and with correspondingly low electrical power consumption.

The exoskeleton according to the invention thus achieves effective, passive, non-actuator or non-motor-based gait support or relief and/or support of the human knee joint concerned, for all desired ranges of movement, including walking on level ground, walking downhill, walking uphill/climbing uphill/climbing stairs, ski touring, walking downhill/climbing down stairs. In addition, the exoskeleton according to the invention is also suitable for applications in outer space.

In the first variant of the invention, the deflection-guide positioning device, which is designed as a device for the resilient positioning of the deflection guide, systematically positions or supports the deflection guide in a compliant manner due to the forces acting in the course of the gear.

The feature that the deflection guide is arranged in front of the joint is also to be understood as an arrangement of the deflection guide in a vertical plane in front of the joint, as viewed in the direction of movement, even if the deflection guide is arranged slightly above or below the height level at which the joint is located.

In addition to a joint arranged on the outside next to the human knee joint, the exoskeleton according to the invention can also have an additional joint arranged on the inside next to the human knee joint, which is arranged in particular at the same height and depth position as the outer joint.

In a view of the exoskeleton from the front, the outer joint, the deflection guide, the elongate spring element with its upper and lower connection points and the deflection-guide positioning device are arranged in particular at a width position which lies on the outer side of the exoskeleton, as seen from the front, and in particular outwardly adjacent to the plane which, again as seen from the front of the exoskeleton, passes through the vertical center plane of the exoskeleton.

According to one embodiment of the invention, the characteristic curve of the elongate spring element is preferably degressive, and the characteristic curve of the deflection spring of the deflection-guide positioning device is preferably progressive.

The springs/spring elements of the deflection-guide positioning device can be designed as classic steel springs, but composite springs, elastomer springs, air springs or other spring elements can also be provided.

The joint that pivotably connects the upper structural part to the lower structural part can be designed as a single joint, a double joint or a spring joint.

The deflection point and thus the deflection-guide positioning device is preferably arranged as centrally as possible between the upper and lower connection points of the elongate spring element.

According to a first embodiment, the deflection-guide positioning device is designed as a spring, in particular a tension spring, which is fixed with its front fixing point to a front, lower section of the upper structural part and which is fixed with its rear fixing point to the deflection guide. In particular, the spring with its front and rear fixing point and the deflection guide are arranged essentially in front of the joint.

According to a first embodiment, the deflection-guide positioning device is designed as a spring, in particular a compression spring, which is fixed with its front fixing point to the deflection guide and which is fixed with its rear fixing point to a lower section of the upper structural part adjacent to the joint. In particular, the rear fixing point of the spring and the deflection guide are arranged substantially in front of the joint.

Such embodiments are simple and inexpensive to manufacture, compact in design and, according to the first variant of the invention, they permit an effective dynamic change in the distance between the deflection guide and the joint with a corresponding pivoting of the upper and lower structural part, or, in the second variant of the invention, an individual adjustment of the desired distance between the deflection guide and the joint to the desired distance.

According to an alternative embodiment, the deflection-guide positioning device is designed as an arrangement consisting of a lever element and a spring element. The lever element can have a first, in particular shorter leg, and a second, in particular longer leg and a pivot point, which is rotatably mounted on a lower, lateral section of the upper structural part. The deflection guide can be formed or arranged at a lower coupling point of the lever element, which is arranged in particular at the lower end of one of its two legs, in particular at the lower end of its longer leg. The lever element can be coupled to the spring element with an upper coupling point, which is arranged in particular at the upper end of the other of its two legs, in particular at the upper end of its shorter leg. The spring element can be fixed to the lever element with its lower fixing point and fixed to a lateral section of the upper structural part with its upper fixing point.

According to a further embodiment, the deflection-guide positioning device is designed as an arrangement comprising a lever element, a cable/rope and a spring element. The lever element can have a first, in particular shorter leg and a second, in particular longer leg and a pivot point, which is rotatably mounted on a lower, lateral section of the upper structural part. The deflection guide can be formed or arranged at the lower coupling point of the lever element, which is arranged in particular at the lower end of one of its two legs, in particular at the lower end of its longer leg. The lever element can be coupled to the cable/rope with its upper coupling point, which is arranged in particular at the upper end of the other of its two legs, in particular at the upper end of its shorter leg. The spring element can be fastened with its lower end to the upper end of the cable/rope, and it can be fixed with its upper fixing point to an upper section of the upper structural part. The lower end of the cable/rope can be fastened to the lever element, in particular to its upper connection point, which is arranged in particular at the upper end of one of its two legs, in particular at the upper end of its shorter leg.

Both embodiments are effective, inexpensive to manufacture and compact in design. The space required by the deflection-guide positioning device in the area of the knee in front of the joint is very small. This means that the exoskeleton can be very slim in the area of the knee.

In the first variant of the invention, both embodiments allow a dynamic change in the distance between the deflection guide and the joint when the upper and lower structural parts are pivoted in relation to each other and thus a dynamic adjustment of the acting moment and a dynamic adjustment of the degree of relief and/or support.

In the second variant of the invention, the distance between the deflection guide and the joint can be easily adjusted to the desired distance in both embodiments.

According to a further embodiment, the pivot point of the lever element, in relation to the upper structural part, is arranged in a plane behind the upper fixing point of the spring element. The two legs of the lever element, as seen from the pivot point, are in particular directed diagonally forwards and upwards. The spring element is designed in particular as a tension spring.

It is preferable for the spring element to be suspended in such a way that the spring action is guided exactly through the joint center/joint at the end stop. The spring element must therefore be attached a little further down on the upper structural part.

According to a further, alternative embodiment, the pivot point of the lever element, in relation to the upper structural part, is arranged in a plane in front of the upper fixing point of the spring element. The two legs of the lever element are directed diagonally backwards and diagonally downwards, as seen from the pivot point. The spring element is designed in particular as a compression spring.

It is preferable for the spring element to be suspended in such a way that the spring action is guided exactly through the joint center/joint at the end stop. The spring element must therefore also be attached a little further down on the upper structural part.

According to a further embodiment, at least one stop is also provided for the lever element, which limits the rotary movement of the lever element. This stop can be designed to be adjustable in order to adjust the effect of the deflection-guide positioning device. In particular, the stop for the lever element can be arranged in such a way that one of the two legs of the lever element, in particular the longer leg of the lever element, strikes against the stop during its movement to the rear, thus limiting the rotary movement of the lever element. The stop can be arranged in particular on a lower, lateral section of the upper structural part, in particular above the joint.

The rotary movement of the lever element can be limited by such a stop and thus the deflection-guide positioning device can be individually adjusted to the user. In particular, the degree of relief and/or support can be limited in one direction in the first variant of the invention.

According to a further embodiment, the at least one joint has an upper and a lower hinge part. The upper hinge part of the outer joint can be formed with two spaced plates, and the deflection guide and the lower part of the lever element can be guided between the spaced plates and covered laterally by them when the upper and lower structural parts are pivoted in relation to one another.

Such an embodiment is particularly space-saving. The central area of the elongate spring element, together with the deflection guide, can be guided between the spaced plates when the upper and lower structural parts are pivoted towards each other and covered laterally by them, which also provides protection against accidental jamming.

According to a further embodiment, the deflection-guide positioning device is designed as an adjustment cable with deflection. The adjustment cable is designed in particular as a cable-spring combination. The adjustment cable can be fastened with its upper connection point to a section of the upper structural part, wherein the upper connection point of the adjustment cable can be arranged in particular in front of the upper connection point of the elongate spring element, in relation to the upper structural part. The adjustment cable can be guided via an adjustment cable deflection point; wherein the adjustment cable deflection point is arranged in particular on a lower section of the upper structural part and positioned in front of the deflection guide of the cable element. The adjustment cable can be fastened to the deflection guide with its lower connection point, coming from the front.

This embodiment is effective, inexpensive to manufacture and compact. The space required by the deflection-guide positioning device in the area of the knee in front of the joint is very small. This means that the exoskeleton can be very slim in the area of the knee.

In the first variant of the invention, this embodiment permits a dynamic change in the distance between the deflection guide and the joint when the upper and lower structural parts are pivoted in relation to one another and thus a dynamic adjustment of the acting moment and a dynamic adjustment of the degree of relief and/or support.

In the second variant of the invention, the distance between the deflection guide and the joint can be easily adjusted to the desired distance.

According to a further embodiment, the deflection-guide positioning device, which is designed as a device for adjusting the deflection guide, is also designed in such a way that the deflection guide can be locked in at least one position at a distance from the joint. In particular, the deflection guide, the spring, the spring element or the adjustment cable can be locked in at least one selectable locking position.

This embodiment relates to the second variant of the invention. As a result, the deflection guide and thus the deflection point/the deflection path of the elongate spring element can be adjusted or locked by a user to a specific distance from the joint. Locking can be performed by a corresponding latching mechanism or a fixing mechanism. In particular, the deflection guide, the spring element or the adjustment cable can be locked in at least one of a plurality of selectable locking positions.

According to a further embodiment, the deflection-guide positioning device is designed as an actuator. The actuator is arranged in particular on a lateral lower section of the upper structural part. It can be designed as a hydraulic actuator, in particular as a hydraulic cylinder, as an electric actuator, in particular as a torque motor, as a pneumatic actuator, as a piezo-electric actuator or as a passive actuator. Furthermore, a pressure accumulator and control valves can be provided.

By means of such an actuator, according to the first variant of the invention, both the distance between the deflection guide and the joint can be resiliently and/or dynamically adjusted during the corresponding pivoting of the upper and lower structural part and thus of the human upper and lower leg in relation to each other, resulting in a corresponding dynamic adjustment of the acting moment and a corresponding dynamic adjustment of the degree of relief and/or support. It is also possible, according to the second variant of the invention, to adjust the distance between the deflection guide and the joint to a desired distance by means of the actuator and to lock the deflection guide at this distance from the joint, which results in the advantages stated above with reference to the second variant of the invention.

According to a further embodiment, an adjustment device for the upper connection point or the lower connection point or a tensioning device for the elongate spring element is also provided at the upper connection point or at the lower connection point.

According to a further embodiment, the elongate spring element and/or the deflection-guide positioning device is surrounded at least in certain areas by a protective sheath, a protective tube or a cover.

According to a further embodiment, the upper and lower structural parts are each cuff-like or half-shell-shaped with adjustable straps so that they can be detachably attached to a leg. In particular, the upper structural part has a larger diameter than the lower structural part.

The invention is explained in more detail below using exemplary embodiments with reference to the enclosed figures.

### BRIEF DESCRIPTION OF THE DRAWINGS

FIG. 1 shows a diagram of an exoskeleton 2 having an upper structural part 4 and a lower structural part 6, which are pivotably connected to each other by means of a single joint 8 (FIG. 1(a)), by means of a double joint 8 (FIG. 1(b)) and by means of a spring joint 8 (FIG. 1(c)), and having an elongate spring element 10 whose deflection point 16 can be moved forwards and backwards.

FIG. 2 shows a diagram of the exoskeleton 2 from FIG. 1(a) with the relevant parameters marked.

FIG. 3 shows a diagram of an exoskeleton 2 with the elongate spring element 10 and with a deflection-guide positioning device 18 with tension spring 20, according to a first exemplary embodiment of the present invention, in two different positions (FIGS. 3(a) and (b)), and a diagram of an exoskeleton 2 according to the invention with a deflection-guide positioning device 18 with compression spring 26, according to a second exemplary embodiment of the present invention, in two different positions (FIGS. 3(c) and (d)).

FIG. 4 shows a diagram of an exoskeleton 2 with a deflection-guide positioning device 18 comprising a lever element 28, a cable 40 and a tension spring 42, according to a third exemplary embodiment of the present invention, in two different positions (FIGS. 4(a) and (b)) and a diagram of an exoskeleton 2 with a deflection-guide positioning device 18 comprising a lever element 28, a cable 40 and a compression spring 48, according to a fourth exemplary embodiment of the present invention, in two different positions.

FIG. 5 shows a schematic view from the front of two exoskeletons 2 with a deflection-guide positioning device 18 according to the third exemplary embodiment of FIG. 4 on a right and on a left human leg (FIG. 5(a)), and a schematic view from the front of two exoskeletons 2 according to the invention on a left and right human leg, according to the third exemplary embodiment of FIG. 4, wherein additionally two struts which can be pivoted towards each other about an inner joint are shown on the inner sides of the two exoskeletons 2.

FIG. 6 shows a diagram of an exoskeleton 2 with a deflection-guide positioning device 18 with an adjustment cable 30 with spring 32, according to a fifth exemplary embodiment of the present invention, in two different positions (FIGS. 6(a) and (b)).

FIG. 7 shows two perspective views of a prototype exoskeleton 2 according to a sixth exemplary embodiment of the present invention, in which the deflection-guide positioning device 18 comprises a lever element 28 and a spring 48, in two different positions (FIGS. 7(a) and (b)).

### DETAILED DESCRIPTION

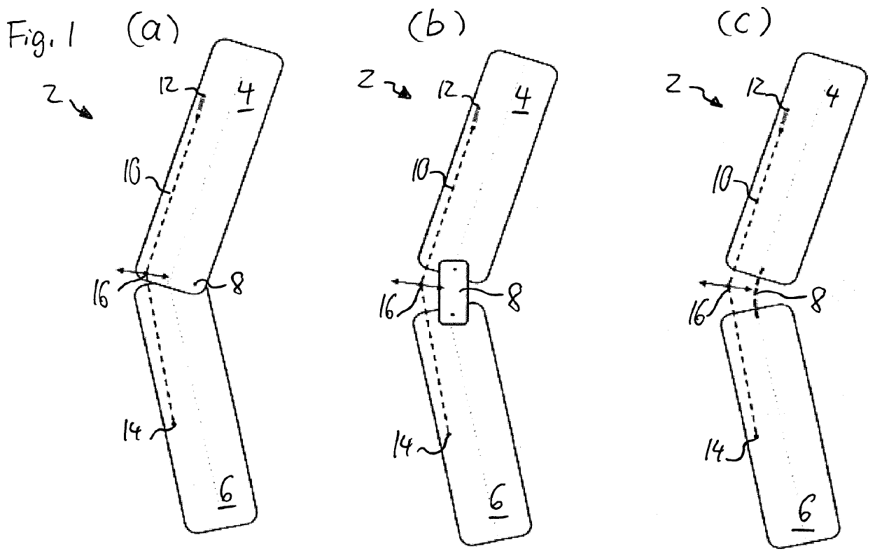

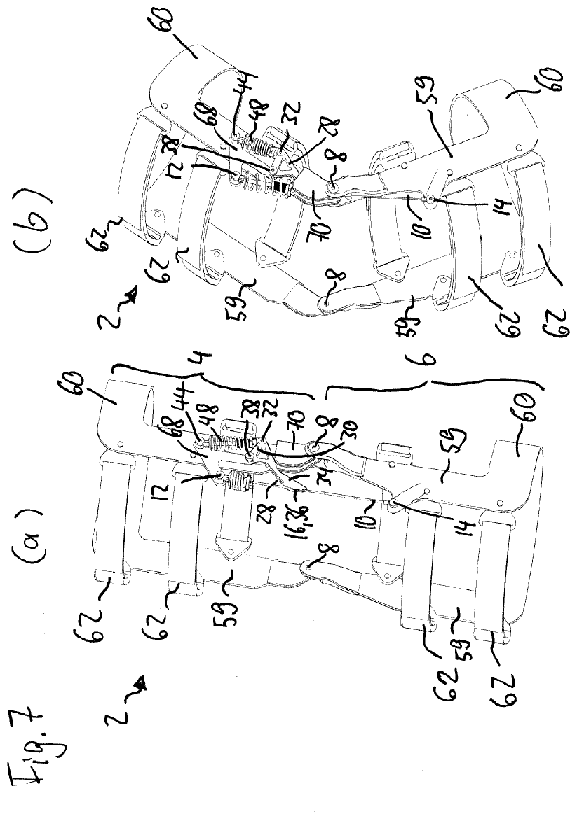

The exoskeletons 2 shown in FIGS. 1 to 7 have an upper structural part 4 for attachment to a human thigh and a lower structural part 6 for attachment to a human lower leg, which are connected to each other by means of at least one joint 8 arranged laterally on the outside next to the human knee joint. In FIGS. 1 to 4 and 6, the structural parts 4 and 6 are shown in the form of rectangles for simplification; in practice, these can have a half-shell, lateral struts and/or fastening straps by means of which the upper and lower structural parts 4, 6 can be fastened to the respective human upper and lower leg, as can be well imagined with reference to FIG. 7.

The joint 8 can be arranged in various ways and in various positions between the upper structural part 4 and the lower structural part 6. In FIG. 1(a) and FIGS. 2 to 7, the joint 8 is designed as a single joint. In FIG. 1(b), the joint is designed as a double joint, with a pivotable plate, wherein the lower joint of the double joint is pivotably connected to the lower structural part 6 and the upper joint of the double joint is pivotably connected to the upper structural part 4. In FIG. 1(c), the joint is designed as a spring joint 8.

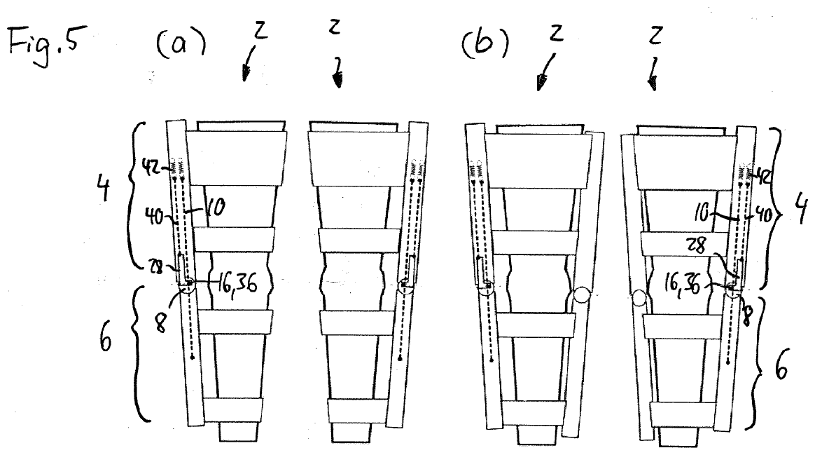

In a simple embodiment, it is sufficient if a joint is arranged laterally, preferably on the outside next to the human knee joint, as can be clearly seen, for example, in the front view of FIG. 5(a). To increase stability, a further joint can also be provided on the inside, disposed on the inner side of the human knee joint, which pivotably connects the upper structural part 4 and the lower structural part 6, as can be clearly seen in the front view of FIG. 5(b) and in the views of the prototype in FIG. 7.

The exoskeletons shown in FIGS. 1 to 7 further comprise an elongate spring element 10, which extends between an upper connection point 12, which is arranged in particular at a lateral, front region of the upper structural part 4, and a lower connection point 14, which is arranged in particular at a lateral, front region of the lower structural part 6, and which is guided via a deflection guide, which defines a deflection point or a deflection path for the elongate spring element 10. The elongate spring element 10 can be designed as an elongate elastomer spring, as an elastic band or as a band-spring combination. The latter variant is shown in FIGS. 1 to 7, wherein the spring of the band-spring combination is arranged at the upper end in each case and is connected with its upper end, which represents the upper connection point 12 of the band-spring combination as a whole, to a suitable point or a suitable mounting of the upper structural part 4. The band is attached to the lower connection point of the spring, which extends via the deflection point 16 to the lower connection point 14 on the lower structural part 6.

The exoskeletons shown in FIGS. 1 to 7 also have a deflection-guide positioning device 18, wherein there are two invention variants here.

In the first variant of the invention, the deflection-guide positioning device 18 is configured as a device for resiliently positioning the deflection guide 16 and is configured such that the deflection guide 16 is resiliently positionable in the direction of increasing the distance and decreasing the distance between the deflection guide 16 and the joint 8, as viewed substantially in the direction forward from the joint 8, for dynamically adjusting the degree of relief and/or support of the human knee joint.

In the second variant of the invention, the deflection-guide positioning device 18 is configured as a device for adjusting the deflection guide 16 and is configured such that the distance between the deflection guide 16 and the joint 8, as viewed substantially in the direction from the joint 8 towards the front, is adjustable for adjusting the degree of relief and/or support of the human knee joint. In the second variant of the invention, the deflection guide 16 can further be locked in at least one position at a distance from the joint 8.

In FIGS. 1(a), (b) and (c), only the spring direction, for the first invention variant, or the adjustment position direction, for the second invention variant, is shown by means of a double arrow. In this direction, the deflection guide 16 and with it the deflection point/the deflection path, which for the sake of simplicity are marked with the same reference sign 16, can move resiliently in the first invention variant or the deflection guide 16, in the second invention variant, can be locked in a desired setting position. In the following, deflection point 16 is also understood to mean a deflection path.

In this case, the deflection guide 16 and the deflection point 16 are located at a position in front of the joint 8 and essentially at the height position of the joint 8, wherein it is also conceivable here that the deflection guide 16 and the deflection point 16 are offset upwards or downwards in relation to the joint 8, as long as they are only arranged in a vertical plane in front of the joint 8.

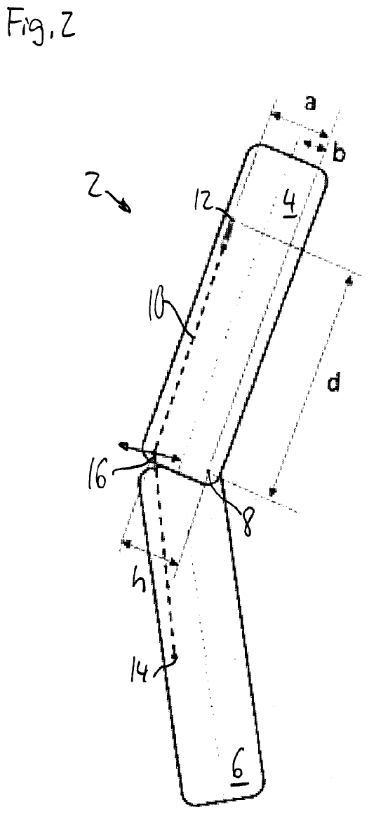

In FIG. 2, the parameters a, b, d, h and (a-h) are defined as follows:
a: Distance of the upper connection point 12 of the elongate spring element 10 to the joint 8;
h: Distance of the deflection point 16/the deflection guide 16 to the joint 8;
a-h: Distance of the upper connection point 12 of the elongate spring element 10 to the deflection point 16/the deflection guide 16;
b: Back offset of the spring joint 8.

All these variables are related to the direction perpendicular to the vertical plane through the exoskeleton 2 or through the upper structural part 4 of the exoskeleton 2.

The parameter d denotes the distance between the upper connection point 12 of the elongate spring element 10 to the deflection point 16/the deflection guide 16, as viewed in the direction of the plane that extends vertically through the upper structural part 4 of the exoskeleton 2.

In particular, the ratio of a/d determines the basic torque curve, which is modified in accordance with the invention by moving the deflection point 16/the deflection guide 16 along the double arrow, as a result of which the characteristic variable h and also the characteristic variable a-h change.

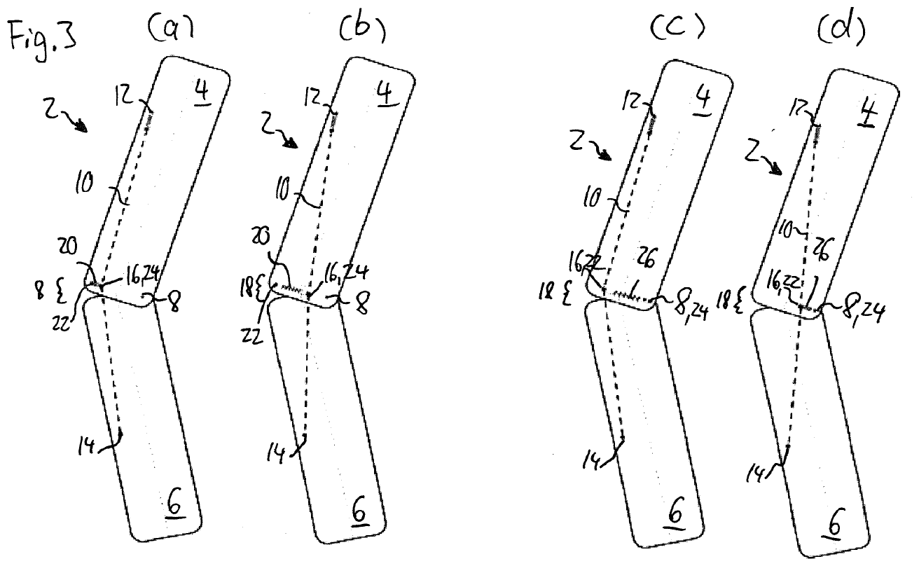

In the exemplary embodiment of FIGS. 3(a) and (b), the deflection-guide positioning 18 is designed as a tension spring 20, which is fixed with its front fixing point 22 to a front, lower section of the upper structural part 4 at a position in front of the joint 8 and which is fixed with its rear fixing point 24 to the deflection guide 16. In this case, the tension spring 20 is arranged with its front fixing point 20 and with its rear fixing point 24 in front of the joint 8, and thus the deflection guide 16 is also arranged in front of the joint 8.

In FIG. 3(a), the tension spring 20 is shown with a small deflection/a small length, so that the total length (a-h) of the tension spring 20 is small, but the distance h between the deflection point 16 and the joint 8 is large. The angle between the lower section and the upper section of the elongate spring element 10 is relatively large, approximately 20-25° in the present drawing, and a correspondingly large supporting moment acts on the human knee joint.

In the illustration of FIG. 3(b), the tension spring 20 is shown with a greater deflection/length, so that the distance (a-h) between the front fixing point 22 and the rear fixing point 24 of the tension spring 20 is greater, and accordingly the distance h between the deflection point 16 and the joint 8 is small. The angle between the lower and the upper section of the elongate spring element 10 is small and is approximately 5° in the illustration in FIG. 3(b). Accordingly, only a small supporting moment acts on the human knee joint.

In the exemplary embodiment of FIGS. 3(c) and (d), the deflection-guide positioning device 18 is formed as a compression spring 26 which is fixed with its front fixing point 22 to the deflection guide 16 and which is fixed with its rear fixing point 24 to a lower portion of the upper structural part 4 adjacent to the joint. The front fixing point 22 of the compression spring 26 and thus the deflection guide 16 is arranged in front of the joint 8. The rear fixing point 24 of the compression spring 26 can be arranged slightly in front of the joint, for example at the level of the joint 8 or even slightly behind the joint 8.

When it is mentioned in the present description that one of the connection points of the spring is arranged in front of or behind the joint 8, it is important that the vertical plane in relation to the exoskeleton 2, in which the respective fixing or connection point is located, is arranged in a corresponding position in front of or behind the vertical plane that extends through the joint 8. The respective anchoring or connection point therefore does not have to be exactly at the height of the joint 8, but can be a little above or below the horizontal plane that extends through the joint 8.

In the illustration of FIG. 3(c), the compression spring 26 is shown with a large deflection/a large overall length. Accordingly, the distance h between the deflection point 16 and the joint 8 is relatively large, and the lower and upper sections of the elongate spring element 10 are arranged at a relatively large angle, in the illustration at about 20-25° to each other, accordingly a relatively large supporting moment acts on the human knee joint. In the illustration of FIG. 3(d), the compression spring 26 is shown with a small deflection/a small overall length, i.e. in a compressed state. Accordingly, the distance h between the deflection point 16 and the joint 8 is small, and the upper and lower sections of the elongate spring element 10 are arranged at a small angle to each other or even lie in a line, as shown in FIG. 3(d), so that there is only a small or no supporting moment for the human knee joint.

In the first variant of the invention, the deflection point 16 is resiliently mounted by the tension spring 20 or by the compression spring 26, resulting in a dynamic torque curve. In particular, it may be preferred that the supporting moment is reduced/damped by the tension spring 20 or the compression spring 26 when the upper structural part 4 is pivoted towards the lower structural part 6.

In the second variant of the invention, the tension spring 20 or the compression spring 26 or the deflection guide 16 is locked in the desired length and position by a suitable locking or engaging device, and the distance between deflection point 16/deflection guide 16 and joint 8 remains essentially constant for the movement process.

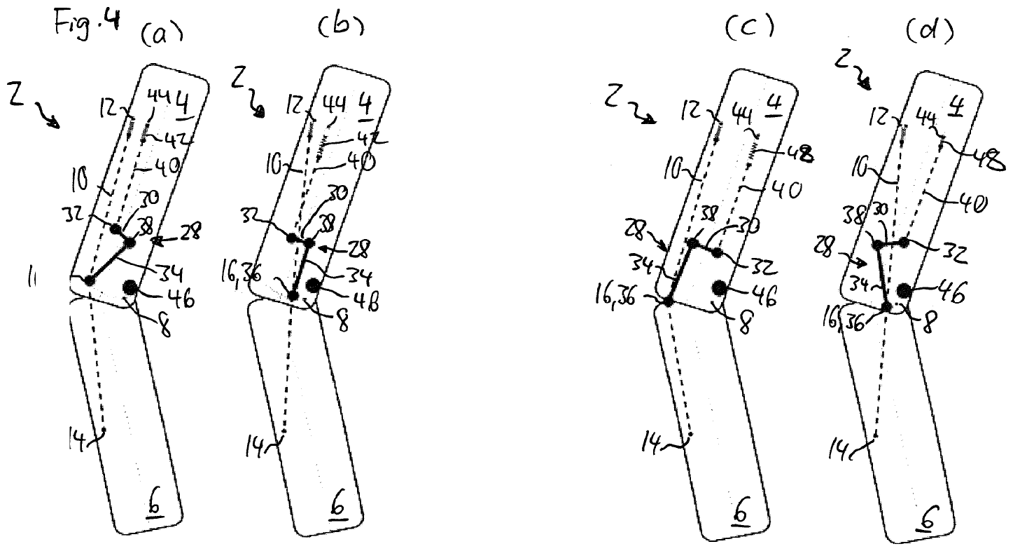

In the exemplary embodiment shown in FIGS. 4(a) and (b), the deflection-guide positioning device 16 is designed as an arrangement comprising a lever element 28, a cable 40 and a tension spring 42. The lever element 28 comprises a first shorter leg 30 and a second longer leg 34, which are arranged in particular at right angles to one another. The pivot point 38 is arranged at their transition, by means of which the lever element 28 is rotatably mounted on a lower lateral section of the upper structural part 4. At the lower end of the longer second leg 34, the lever element 28 is coupled to the deflection guide 16 via the deflection-guide coupling point 36 or the deflection guide 16 is formed there. The elongate spring element 10 extends over or through this deflection guide 16. A cable coupling point 32 is formed at the upper end of the shorter leg 30, at which the cable 40 is fixed and from which the cable 40 extends to the tension spring 42. The cable is attached to the lower end of the tension spring 42. With its upper end, the tension spring 42 is fixed to an upper section of the upper structural part 4.

As can be clearly seen from the illustrations in FIGS. 4(a) and (b), the pivot point 38 of the lever element 28 is arranged in relation to the upper structural part 4 in a vertical plane behind the upper fixing point 44 of the spring element 42, but in front of the vertical plane through the joint 8. Furthermore, it is easy to see that the upper fixing point 44 of the spring element 42 is arranged in a vertical plane behind the plane formed by the upper connection point 12 of the elongate spring element 10.

In the illustration of FIG. 4(a), the distance between the cable fixing point 44 and the cable coupling point 32 has been slightly shortened by a corresponding contraction/shortening of the tension spring 42. As a result, the lever element 28 has been pulled slightly upwards with its shorter leg 30 and pivoted clockwise about the pivot point 38, so that the deflection-guide coupling point 36 has been pivoted a little diagonally to the left, which in turn has increased the distance between the deflection guide 16 and the joint 8. Accordingly, the angle between the upper and lower sections of the elongate spring element 10 has also increased, resulting in a greater supporting moment on the human knee joint.

In the present purely exemplary and non-limiting representation, this angle is approximately 20-25°. In the position of FIG. 4(b), the distance between the cable fixing point 44 and the cable coupling point 32 has been increased by a corresponding pulling apart of the tension spring 42. Accordingly, the lever element 28 has been pivoted counterclockwise so that the shorter leg 30 is approximately perpendicular to the vertical axis of symmetry through the upper structural part 4 and accordingly the longer leg 34 is directed downwardly substantially parallel to this vertical axis of symmetry. The distance between the deflection point 16 and the joint 8 is therefore small and there is only a small angle between the upper and the lower section of the elongate spring element 10 or no angle at all, so that there is little or no moment support for the human knee joint.

The shorter leg 30 of the lever element 28 is arranged in particular in a plane at a distance from the vertical plane, which extends from front to rear in a plan view of the exoskeleton 2, as can be clearly seen in FIG. 5, so that in the pivoting position according to FIG. 4(b) the shorter leg 30 is arranged laterally next to the elongate spring element 10 and does not touch the latter when it passes under the elongate spring element 10.

The exoskeleton 2 according to the present exemplary embodiment further comprises a stop 36 for the lever element 26, in particular for the longer leg 34, which limits the rotational movement of the lever element 28, in particular towards the rear. This stop 46 is arranged in particular on a lower lateral section of the upper structural part 4 above the joint 8. In particular, this stop 46 can also be designed to be adjustable in order to limit the rearward pivoting of the deflection guide 16 and to adapt the torque range that can be achieved by the exoskeleton 2 to the respective user.

In the exemplary embodiment shown in FIGS. 4(c) and (d), the deflection-guide positioning device 18 is also designed as an arrangement comprising a lever element 28, a cable 40 and a spring element 48, wherein, in contrast to the exemplary embodiment of FIGS. 4(a) and (b), the spring element 48 is designed as a compression spring. Identical elements are marked with the same reference signs and are not described separately again here.

Due to the different designs of the spring element, i.e. compression spring 48 instead of tension spring 42, the lever element 28 is designed/arranged differently. In the exemplary embodiment of FIGS. 4(a) and (b), the lever element 28 has the appearance of a large L mirrored around the pivot point 38 or a hook pointing to the left. In the exemplary embodiment of FIGS. 4(c) and (d), the lever element 28 has the appearance of an inverted L mirrored along a horizontal axis or a rearwardly directed hook.

In the exemplary embodiment shown in FIGS. 4(c) and (d), the pivot point 38 of the lever element 28 lies in a vertical plane, again with respect to the upper structural part 4 of the exoskeleton 2, in front of the vertical plane of the cable fixing point 44 and behind the vertical plane of the upper connection point 12 of the elongate spring element 10. The upper cable fixing point 44 is located in a vertical plane that extends directly in front of the vertical plane through the joint 8 and is offset to the rear with respect to the vertical plane in which the cable fixing point 44 of the exemplary embodiment of FIGS. 4(a) and (b) is located.

In the exemplary embodiment of FIGS. 4(c) and (d), the cable 40 is to be designed to be particularly rigid, for example as a steel cable and, optionally, provided with a corresponding guide, not shown here, in order to be able to transmit the effect of the compression spring 48 to the lever element 28.

In the position of FIG. 4(c), the compression spring 48 is shown in its longer, pushed apart state 48, so that the distance between the cable fixing point 44 and the cable coupling point 32 is relatively large. Accordingly, the shorter leg 30 extends in a plane approximately perpendicular to the vertical plane of symmetry through the upper structural part 4 or is even inclined a little to the lower right in a clockwise direction with respect thereto. Accordingly, the longer leg 34 extends in this vertical plane of symmetry or is even inclined a little to the left from this plane in a clockwise direction. Accordingly, the distance between the deflection guide 16 and the joint 8 is relatively large, and the angle between the lower and upper sections of the elongate spring element 10 is also large, and is approximately 25-30° in the present purely exemplary and non-limiting embodiment. In contrast, in the position shown in FIG. 4(d), the compression spring 48 is compressed a little and has a shorter length than in the position shown in FIG. 4(c). Accordingly, the distance between the cable fixing point 44 and the cable coupling point 32 is smaller, and the lever element 28 is pivoted with its shorter leg 30 and with its longer leg 34 in a counterclockwise direction diagonally upwards. As a result, the distance between the deflection guide 16 and the joint 8 is so small that there is only a small angle between the upper and lower sections of the elongate spring element 10 or no angle at all and, accordingly, only little or no moment support acts on the human knee.

In the first variant of the invention, the deflection guide 16 is resiliently mounted by the deflection-guide positioning device 18 shown in FIG. 4. In the second variant of the invention, the distance between the deflection guide 16 and the joint 8 is initially set to a desired distance and the deflection-guide positioning device 18 is locked at this distance, for example by locking the lever element 28 with a suitable locking mechanism.

FIGS. 5(a) and (b) show exoskeletons 2, in which the deflection-guide positioning device 18 is designed as shown in FIG. 4, schematically from the front. It can be clearly seen that the tension spring 42, the cable 44 and at least partially the lever element 28 are arranged in a plane seen from the front to the rear laterally outside and next to the plane in which the elongate spring element 10 is arranged. Furthermore, the upper structural part 4 and the lower structural part 6 are each designed with a lateral, outer strut and with fastening straps, in each case two fastening straps for the human upper leg on the upper structural part 4 and two fastening straps for the lower leg on the lower structural part 6. In the representation of FIG. 5(b), struts are also provided on the inside, each of which can be pivoted towards one another by means of an additional hinge located on the inside.

In the embodiments of FIGS. 4 and 5, it is preferable if the lever element 28 is positioned at the end stop 48 in such a way that the spring action is guided exactly through the joint center point/joint 8. In the other positions, the positioning is not so central and should be as central as possible between the spring connection points 12 and 14. This can be defined by the choice of the pivot point 38 of the lever element 28.

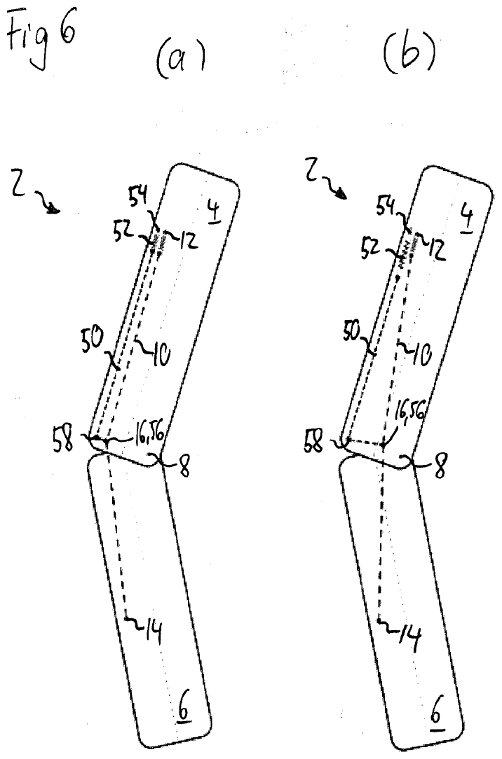

In the exemplary embodiment shown in FIGS. 6(a) and (b), the deflection-guide positioning device 18 is designed as an adjustment cable 50 with deflection. In particular, the adjustment cable 52 is designed as a cable-spring combination, with an upper tension spring 52 and a cable 50 connected to it at the bottom. The upper tension spring 52 is attached with its upper connection point 54 to an upper front section of the upper structural part 6. The adjustment cable 50 is attached to the lower end of the upper tension spring 52. This is guided via an adjustment cable deflection point 58, which is positioned on a lower, lateral section of the upper structural part 4 in front of the deflection guide 16 of the elongated cable element 50 and thus also in front of the joint 8.

The upper connection point 54 of the cable-spring combination of the adjustment cable 50 is arranged in front of the upper connection point 12 of the spring element 10.

The adjustment cable 50 is attached to the deflection guide 16 with its lower connection point 36 coming from the front. In the position shown in FIG. 6(a), the tension spring 52 is shown in its contracted, shortened state. Accordingly, the distance of the adjustment cable 50 between the adjustment cable deflection point 58 and the deflection guide 16 is small, and accordingly the distance h between the deflection guide 16 and the joint 8 is large. The angle between the lower and the upper section of the elongate spring element 10 is relatively large and is approximately 20-25° in the present purely exemplary and non-limiting representation. A corresponding supporting moment thus acts on the human knee joint.

In the position of FIG. 6(b), the tension spring 52 is shown in its extended, pulled-apart state. Accordingly, the distance of the section of the adjustment cable 50 between the adjustment cable deflection point 58 and the deflection guide 16 is relatively large, and the distance h between the deflection guide 16 and the joint 8 is relatively small. The upper and lower sections of the elongate spring element 10 are arranged at a slight angle of approximately 5° to each other. This means that a correspondingly low supporting moment acts on the human knee joint.

In the first variant of the invention, the deflection point 16 is resiliently mounted by means of the adjustment cable 50 designed as a cable-spring combination, and this results in dynamic torque adjustment when pivoting from the upper structural part 4 to the lower structural part 6. In particular, this results in damping and softening of the acting torque when pivoting from the upper structural part 4 to the lower structural part 6.

In the second variant of the invention, the distance between the adjustment cable deflection point 58 and the deflection guide 16 and thus the distance between the deflection guide 16 and the joint 8 is set at the beginning, for example by locking the deflection guide 16 accordingly. The distance between deflection guide 16 and joint 8 then remains constant during the subsequent movement sequence.

In the prototype illustration shown in FIG. 7, the upper structural part 4 and the lower structural part 6 are each designed with two lateral struts 59, which are connected to each other at the upper and lower ends respectively by means of a half-shell 60. In particular, the lateral struts 59 and the half-shells 60 are each designed as a common injection-molded plastic part. In order to be able to securely hold the human thigh or the human lower leg therein, two front fastening straps 62 and one rear fastening strap 62 are arranged on the lateral struts 59.

At the upper and lower ends of the lateral struts 59 of the upper and lower structural parts 4, 6, hinge parts are formed, which are in particular made of metal. Interacting hinge parts are each pivotably connected to one another by means of an inner and outer joint 8.

In the exemplary embodiment of FIGS. 7(a) and (b), the deflection-guide positioning device 18 is designed as an arrangement comprising a lever element 28 and a spring element 48, in particular a compression spring.

The lever element 28 has a curved shape but, like the lever element 28 in FIGS. 4 and 5, has a shorter leg 30 and a longer leg 34. At the front, distal end of the longer leg 34, the deflection guide 16 is designed as a notch-like receptacle for the elongate spring element 10. The lever element 28 is pivotable about the pivot point 38, which is located in a vertical plane in front of the upper connection point 54 of the compression spring 48. The upper connection point 54 of the compression spring 48 is attached to a lateral outer region of the outer strut 59 of the upper structural part 4, in particular to an attachment piece 58, to which a corresponding attachment for the upper connection point 12 of the elongate spring element 12 is also provided towards the front. With its lower connection point 32, the spring element 48 is coupled to the shorter leg 30 of the lever element 28.

The right upper metallic hinge part 70 is formed with two spaced plates and, when the upper structural part 4 is pivoted to the lower structural part 6 as shown in FIG. 7(b), receives the deflection guide 16 and the front lower portion of the lever element 28 therebetween and covers it from both sides, thereby providing a protective effect and preventing pinching.

The pivot point 38 is also formed on the fastening piece 28 and lies in a vertical plane between the upper connection point 12 of the elongate spring element 10 and the upper connection point 44 of the spring element 48.

In FIG. 7(a), the upper and lower structural parts 4, 6 are not pivoted towards each other. The spring element 48 is shown in a position in which the deflection guide 16 is in contact with the elongate spring element 10, but the latter forms only a small angle or no angle at all between its upper and lower sections, resulting in no or only very little moment support for the human knee joint.

In the illustration in FIG. 7(b), the upper and lower structural parts 4, 6 are pivoted towards each other by an angle of approximately 40°. The spring element 48 is in a shortened, compressed position. However, due to the lever element 28, the angle between the lower and upper sections of the elongate spring element 10 is significantly reduced in comparison and is only in a range of about 20° (purely exemplary and not limiting), resulting in a dynamic and softened moment support for the human knee.

In the first variant of the invention, the deflection guide 16 is resiliently mounted; in the second variant of the invention, it is adjusted to a specific distance between the spring element 28 and the joint 8, for example by means of a locking mechanism of the lever element 28, which is not shown here.

With regard to the further advantages and design options of the exemplary embodiments explained above with reference to the figures, reference is made to the general description section; these are not repeated here.

1. An exoskeleton for relieving and/or supporting a human knee joint, comprising:
an upper structural part for fastening to a human thigh;
a lower structural part for fastening to a human lower leg;
wherein the upper structural part and the lower structural part are pivotably interconnected by means of at least one joint arranged laterally, preferably on the outside next to the human knee joint;
a deflection guide arranged in front of the joint;
an elongate spring element, in particular an elongate elastomer spring, an elastic band, or a band-spring combination, which extends between an upper connection point, which is arranged in particular in a lateral, front region of the upper structural part, and a lower connection point, which is arranged in particular in a lateral, front region of the lower structural part, and which is guided over the deflection guide, which defines a deflection point or a deflection path for the elongate spring element;
wherein a deflection-guide positioning device is provided,
wherein the deflection-guide positioning device is in the form of a device for resiliently positioning the deflection guide and is designed such that the deflection guide can be resiliently positioned in the direction of increasing and decreasing the distance between the deflection guide and the joint, when viewed substantially in the direction from the joint towards the front, in order to dynamically adapt the degree of relief and/or support; or
wherein the deflection-guide positioning device is in the form of a device for adjusting the deflection guide and is designed such that the distance between the deflection guide and the joint, when viewed substantially in the direction from the joint towards the front, can be adjusted in order to adjust the degree of relief and/or support.

2. The exoskeleton according to claim 1, wherein the deflection-guide positioning device is designed as a spring, in particular a tension spring, which is fixed with its front fixing point to a front, lower section of the upper structural part, and which is fixed with its rear fixing point to the deflection guide, and/or
wherein in particular the spring with its front and rear fixing point and the deflection guide are arranged substantially in front of the joint.

3. The exoskeleton according to claim 1, wherein the deflection-guide positioning device is designed as a spring, in particular a compression spring, which is fixed with its front fixing point to the deflection guide and which is fixed with its rear fixing point to a lower section of the upper structural part adjacent to the joint; and/or
wherein in particular the rear fixing point of the spring and the deflection guide are arranged substantially in front of the joint.

4. The exoskeleton according to claim 1, wherein the deflection-guide positioning device is designed as an arrangement consisting of a lever element and a spring element;
wherein the lever element has a first, in particular shorter leg and a second, in particular longer leg and a pivot point, which is rotatably mounted on a lower, lateral section of the upper structural part;
wherein the deflection guide is formed or arranged at a lower coupling point of the lever element, which is arranged in particular at the lower end of one of its two legs, in particular at the lower end of its longer leg;
wherein the lever element is coupled to the spring element by an upper coupling point, which is arranged in particular at the upper end of the other of its two legs, in particular at the upper end of its shorter leg,
wherein the spring element is fixed in particular with its lower fixing point to the lever element, and is fixed with its upper fixing point to a lateral section of the upper structural part.

5. The exoskeleton according to claim 1, wherein the deflection-guide positioning device is designed as an arrangement comprising a lever element, a cable/rope and a spring element;
wherein the lever element has a first, in particular shorter leg and a second, in particular longer leg and a pivot point, which is rotatably mounted on a lower, lateral section of the upper structural part,
wherein the deflection guide is formed or arranged in particular at the lower coupling point of the lever element, which is arranged in particular at the lower end of one of its two legs, in particular at the lower end of its longer leg;
wherein the lever element is coupled to the cable/rope with its upper coupling point, which is arranged in particular at the upper end of the other of its two legs, in particular at the upper end of its shorter leg, and
wherein the spring element is fastened in particular with its lower end to the upper end of the cable/rope and is fixed with its upper fixing point to an upper section of the upper structural part, and wherein the lower end of the cable/rope is fastened in particular to the lever element, in particular to its upper coupling point, which is arranged in particular at the upper end of one of its two legs, in particular at the upper end of its shorter leg.

6. The exoskeleton according to claim 4, wherein the pivot point of the lever element, in relation to the upper structural part, is arranged in a plane behind the upper fixing point of the elongate spring element; and/or
wherein the two legs of the lever element, as seen from the pivot point, are directed diagonally forwards and upwards and diagonally forwards, and/or wherein the spring element is designed as a tension spring.

7. The exoskeleton according to claim 4, wherein the pivot point of the lever element, in relation to the upper structural part, is arranged in a plane in front of the upper fixing point of the elongate spring element; and/or
wherein the two legs of the lever element, as seen from the pivot point, are directed diagonally backwards and diagonally downwards; and/or
wherein the spring element is designed as a compression spring.

8. The exoskeleton according to claim 4, wherein furthermore at least one stop is provided for the lever element, which limits the rotary movement of the lever element,
wherein the stop is designed to be adjustable, in particular, for adjusting the effect of the deflection-guide positioning device;
wherein the stop for the lever element is arranged in particular in such a way that one of the two legs of the lever element, in particular the longer leg of the lever element, strikes against the stop during its movement to the rear, thus limiting the rotary movement of the lever element; and
wherein the stop is arranged in particular on a lower, lateral section of the upper structural part, in particular above the joint.

9. The exoskeleton according to claim 4, wherein the at least one joint has an upper and a lower hinge part;
wherein the upper hinge part of the outer joint is formed with two spaced plates, and the deflection guide and the lower part of the lever element are guided between the spaced plates and covered laterally by them when the upper and lower structural parts are pivoted in relation to one another.

10. The exoskeleton according to claim 1, wherein the deflection-guide positioning device is designed as an adjustment cable with deflection;
wherein the adjustment cable is designed in particular as a cable-spring combination;
wherein the adjustment cable is fastened with its upper connection point to a section of the upper structural part, wherein the upper connection point of the adjustment cable is arranged in particular in front of the upper connection point of the elongate spring element, in relation to the upper structural part;
wherein the adjustment cable is guided via an adjustment cable deflection point; wherein the adjustment cable deflection point is arranged in particular on a lower section of the upper structural part and positioned in front of the deflection guide of the cable element; and/or
wherein the adjustment cable is fastened to the deflection guide with its lower connection point, coming from the front.

11. The exoskeleton according to claim 1, wherein the deflection-guide positioning device, which is designed as a device for adjusting the deflection guide, is further designed in such a way that the deflection guide can be locked in at least one position at a distance from the joint; wherein in particular the deflection guide, the spring, the spring element or the adjustment cable can be locked in at least one selectable locking position.

12. The exoskeleton according to claim 1, wherein the deflection-guide positioning device is designed as an actuator;
wherein the actuator is arranged in particular on a lateral lower section of the upper structural part, and
wherein the actuator is designed in particular as a hydraulic actuator, in particular as a hydraulic cylinder, as an electric actuator, in particular as a torque motor, as a pneumatic actuator, as a piezo-electric actuator, or as a passive actuator.

13. The exoskeleton according to claim 1, wherein the force support can be adjusted by the user by means of the deflection-guide positioning device, in particular to a degree of force support between the foremost position of the deflection guide, at which the force support is at its greatest, and the rearmost position of the deflection guide, at which the force support is at its smallest.

14. The exoskeleton according to claim 5, wherein the pivot point of the lever element, in relation to the upper structural part, is arranged in a plane behind the upper fixing point of the elongate spring element; and/or
wherein the two legs of the lever element, as seen from the pivot point, are directed diagonally forwards and upwards and diagonally forwards; and/or
wherein the spring element is designed as a tension spring.

15. The exoskeleton according to claim 5, wherein the pivot point of the lever element, in relation to the upper structural part, is arranged in a plane in front of the upper fixing point of the elongate spring element; and/or
wherein the two legs of the lever element, as seen from the pivot point, are directed diagonally backwards and diagonally downwards; and/or
wherein the spring element is designed as a compression spring.

16. The exoskeleton according to claim 5, wherein furthermore at least one stop is provided for the lever element, which limits the rotary movement of the lever element;
wherein the stop is designed to be adjustable, in particular, for adjusting the effect of the deflection-guide positioning device;
wherein the stop for the lever element is arranged in particular in such a way that one of the two legs of the lever element, in particular the longer leg of the lever element, strikes against the stop during its movement to the rear, thus limiting the rotary movement of the lever element; and
wherein the stop is arranged in particular on a lower, lateral section of the upper structural part, in particular above the joint.

17. The exoskeleton according to claim 16, wherein the at least one joint has an upper and a lower hinge part;
wherein the upper hinge part of the outer joint is formed with two spaced plates, and the deflection guide and the lower part of the lever element are guided between the spaced plates and covered laterally by them when the upper and lower structural parts are pivoted in relation to one another.

18. The exoskeleton according to claim 4, wherein the deflection-guide positioning device, which is designed as a device for adjusting the deflection guide, is further designed in such a way that the deflection guide can be locked in at least one position at a distance from the joint; wherein in particular the deflection guide, the spring, the spring element or the adjustment cable can be locked in at least one selectable locking position.

19. The exoskeleton according to claim 4, wherein the force support can be adjusted by the user by means of the deflection-guide positioning device, in particular to a degree of force support between the foremost position of the deflection guide, at which the force support is at its greatest, and the rearmost position of the deflection guide, at which the force support is at its smallest.

20. The exoskeleton according to claim 4, wherein the force support can be adjusted by the user by means of the deflection-guide positioning device, in particular to a degree of force support between the foremost position of the deflection guide, at which the force support is at its greatest, and the rearmost position of the deflection guide, at which the force support is at its smallest.
